(19) United States
(12) Patent Application Publication
(10) Pub. No.: US 2007/0218090 A1
(43) Pub. Date: Sep. 20, 2007

(54) DISSOLVABLE STRIP FOR DELIVERY OF AN AGENT

(76) Inventor: Craig Berry, Westport, CT (US)

Correspondence Address: PERMAN & GREEN, 425 POST ROAD, FAIRFIELD, CT, 06824, US

(21) Appl. No.: 11/556,870

(22) Filed: Nov. 6, 2006

Related U.S. Application Data
(60) Provisional application No. 60/783,300, filed on Mar. 17, 2006.
(60) Provisional application No. 60/814,829, filed on Jun. 19, 2006.

(51) Int. Cl. A61K 9/70

(52) U.S. Cl. 424/401; 424/443

(57) ABSTRACT

An article of manufacture includes a substrate having at least one zone on a surface of the substrate and at least one ingredient on a surface of the substrate. The at least one ingredient is associated with a respective one of the at least one zone.

This application claims priority to provisional application number 60/783,300 filed on Nov. 18, 2005 and provisional application number 60/814,829 filed on Jun. 19, 2006.

### BACKGROUND

1. Field

The present embodiments relate to dissolvable strips and, more particularly, to dissolvable strips for delivering an agent.

2. Brief Description of Related Developments

Edible film products (e.g. dissolvable strips) are known. The strips are supple in texture are non-self adhering and are commonly sized so they can be placed on the tongue of a user.

One example of a conventional strip is distributed by Pfizer Consumer Healthcare under the name Listerine® PocketPaks™. The Pfizer strips are distributed in a plastic container. A stack of strips is located within the container so that a user may remove individual strips through a hinged opening in the container. These Pfizer strips as well as other conventional strips may be colored so that the strips are associated with another product produced by a respective company or to be visually appealing to a user.

It would be advantageous to have a strip having colors associated with the ingredients of the strip.

### SUMMARY

In one exemplary embodiment an article of manufacture is provided. The article of manufacture includes a substrate having at least one zone on a surface of the substrate and at least one ingredient on a surface of the substrate. The at least one ingredient is associated with a respective one of the at least one zone.

In another exemplary embodiment, an article of manufacture is provided. The article includes a substrate, at least one active ingredient located on a first side of the substrate and at least one ancillary ingredient located on a second side of the substrate. When the at least one active ingredient is placed in contact with the at least one ancillary ingredient the active ingredient is activated or an activity of the active ingredient is enhanced.

In one exemplary embodiment, an article of manufacture is provided. The article includes a substrate, at least one active ingredient located on a first side of the substrate and at least one ancillary ingredient located on a second side of the substrate. When the at least one active ingredient is placed in contact with the at least one ancillary ingredient the at least one active ingredient is activated or an activity of the at least one active ingredient is enhanced.

### BRIEF DESCRIPTION OF THE DRAWINGS

The foregoing aspects and other features of the present embodiments are explained in the following description, taken in connection with the accompanying drawings, wherein:

FIG. 1 illustrates an article in accordance with an exemplary embodiment;

FIG. 2 illustrates another article in accordance with an exemplary embodiment;

FIGS. 3-5 illustrate articles in accordance with exemplary embodiments;

FIG. 6 illustrates an article in accordance with an exemplary embodiment;

FIG. 7 illustrates another article in accordance with an exemplary embodiment;

FIG. 8 illustrates yet another article in accordance with an exemplary embodiment; and

9A and 9B illustrate an article in accordance with an exemplary embodiment.

### DETAILED DESCRIPTION OF THE EXEMPLARY EMBODIMENT(s)

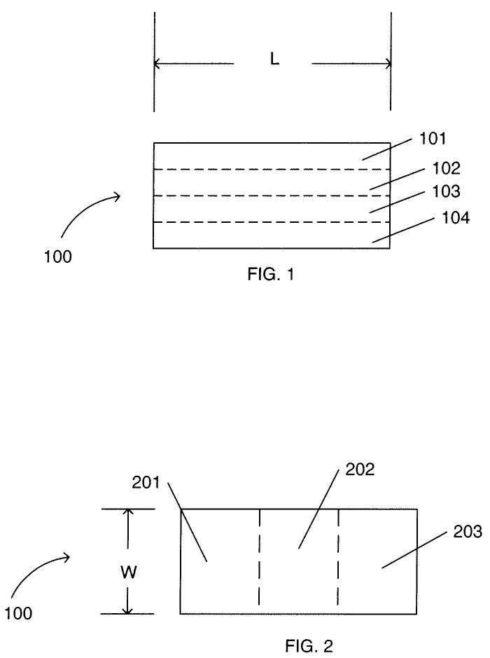

FIG. 1 illustrates a dissolvable substrate or article in accordance with an exemplary embodiment. Although the present embodiments will be described with reference to the exemplary embodiments shown in the drawings and described below, it should be understood that aspects of the invention could be embodied in many alternate forms of embodiments. In addition, any suitable size, shape or type of elements or materials could be used.

The dissolvable substrates or articles described herein, such as the article 100 shown in FIG. 1 may be manufactured from a substrate including a polymeric composition. The polymeric composition may include pullulan and sodium alginate. In alternate embodiments, the strips may be manufactured of any suitable composition(s). The articles described herein may be of any suitable shape and size and have any suitable thickness. The article 100 may also include color bands arranged on the article 100. For example, one or more colors can be arranged in bands 101-104 along the longer dimension L of the article 100. Any suitable colors may be implemented within the bands, for example, the color in band 101 may be yellow, the color in band 102 may be orange, the color in band 103 may be red and the color in band 104 may be green. In alternate embodiments, the same color may be used in more than one band. In another exemplary embodiment, as can be seen in FIG. 2, color bands 201-203 may run crosswise across the narrower dimension W of the article 100. In alternate embodiments, any suitable number of color bands may be arranged in any suitable manner on the article 100. For example, the color bands may be in a diagonal pattern, V-shaped pattern, rainbow shaped pattern, etc.

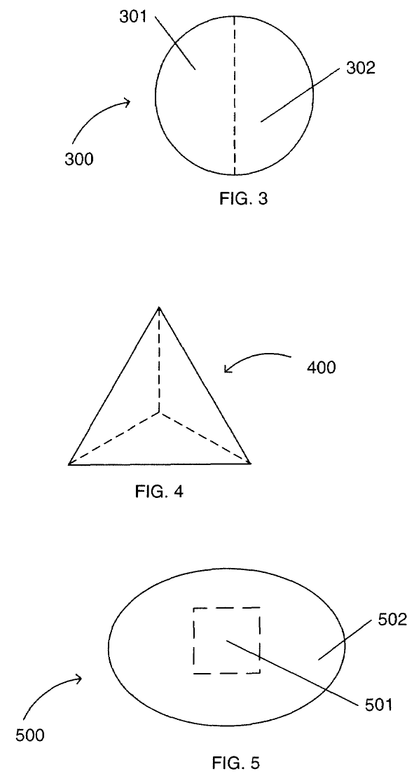

In FIGS. 1 and 2, the article 100 is shown as having a rectangular shape. In alternate embodiments, the article may have any suitable shape. For example, as can be seen in FIGS. 3-5, the article may be a circular article 300, an ovoid article 500 or a triangular article 400. In other alternate embodiments, the article may be shaped like a square, star, an animal, plant or any other suitable shape.

The article 100 may also have any suitable agents, ingredients or formulations (all of which are hereinafter collectively referred to as “ingredients”) arranged on a surface of the article 100. The ingredients may be any suitable formulation related to for example, flavor, fragrance or active ingredients. The active ingredients may be formulations for the treatment of a human or an animal and may include products for treating any suitable ailment or medical condition of a user such as pharmaceutical agents for treating a cough, cold, allergy, sore throat, decongestant, hypertension, headaches etc. The active ingredients may also include formulations that are not medical related such as appetite stimulants and the like. The ingredients may be arranged over the article's entire surface or a section/portion of the surface. The front, back and/or side surfaces of the article 100 may include separate zones, each of which may include a respective ingredient. The ingredient in each zone may or may not be the same as any other zone on the article 100. For example, referring to FIG. 5, the article 500 may have two zones 501, 502 where zone 501 includes a first ingredient and the second zone includes a second ingredient. The second ingredient may or may not be the same as the first ingredient. In alternate embodiments, the article may have any suitable number of zones each having a respective ingredient. The front and back of the article refers to the surfaces of the article having the greatest surface area awhile the sides of the article refers to the surface area of the article pertaining to the articles thickness.

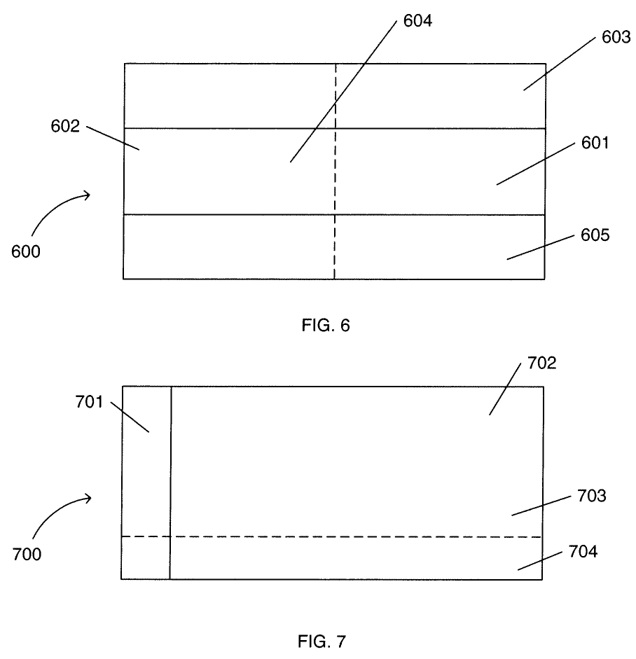

In one embodiment, each zone representing a respective ingredient may be associated with a particular color, which may signify or identify the ingredient. For example, zone 301 of article 300 may include an ingredient for sore throat relief while zone 302 may include an ingredient for suppressing a cough. The sore throat relief in zone 301 may be associated with the color red and the cough suppressant in zone 302 may be associated with the color green. In alternate embodiments, the ingredient zones may be independent of the colors arranged on the article. For example, the article 600 shown in FIG. 6 may have two ingredient zones 601, 602, which in this figure are separated by the dashed line. The color bands 603-605 of the article 600 may traverse more than one ingredient zone such as zones 601, 602.

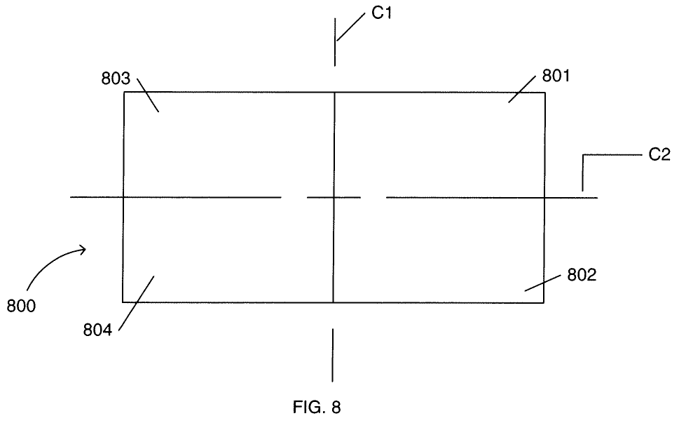

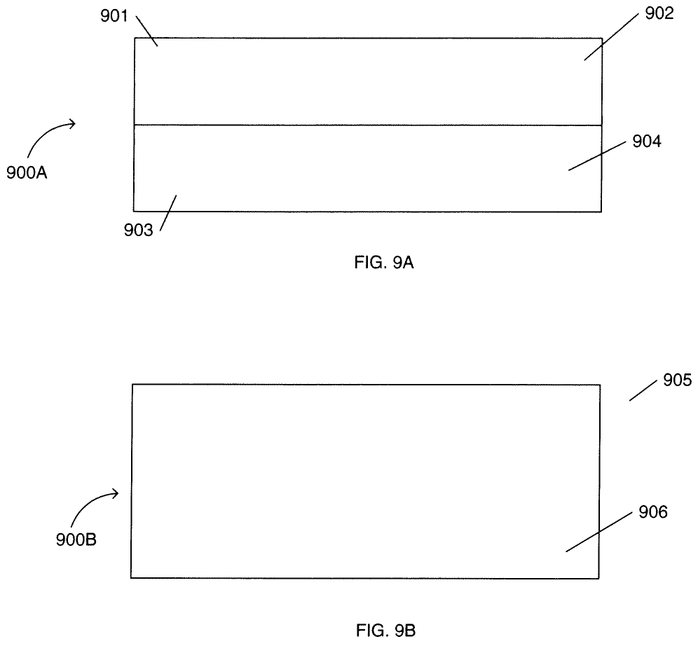

The color bands or ingredient zones may have any suitable shape and any suitable size. For example, in one embodiment, the color bands 603-605 or ingredient zones 601, 602 may be substantially equal in size or they may have different sizes. As can be seen in FIG. 7, one color band 702 or ingredient zone 703 may be larger than the other color bands 701 or ingredient zones 704 and vice versa. In one embodiment, the color bands 801, 803 and/or the ingredient zones 802, 804 may be split at a center point or centerline Cl of the article 800 as shown in FIG. 8. In alternate embodiments, the color bands and/or ingredient zones may be split along centerline C2 or across a diagonal of the article 800.

In one embodiment, each side of the article may include one or more separate ingredients. For example, one side of the article 900A may have an ingredient for sore throat relief and the other side of the article 900B may have another ingredient for relief of another symptom such as a cough suppressant, anti-histamine or decongestant. In another embodiment, each side of the article 900A, 900B may contain one or more ingredient zones. For example, a first side 900A may include a first zone 901 having ingredient “A” and a second ingredient zone 903 having ingredient “B”. The second side of the article 900B may include a third zone 905 having active ingredient “C”. In alternate embodiments, each side of the article may include any suitable number of ingredient zones and corresponding ingredients within those zones.

In alternate embodiments, one side of the article may have an active ingredient while the other side of the article may have an ancillary factor to the active ingredient, such that when the two components are placed in contact, the active ingredient is activated or its activity enhanced. In other alternate embodiments, the two sides may have different colors related to the level of activity of an active ingredient. In still other alternate embodiments, the sides may not have any active ingredient but may contain different flavor or odor components, lending a heightened organoleptic effect by releasing complimentary flavors or a flavor and complementary fragrance.

In one embodiment, each of the ingredient zones may have a color that is associated with the respective ingredient. For example, ingredient “A” in zone 901 may be associated with the color red while ingredient “B” in zone 903 may be associated with the color green. In alternate embodiments, the ingredient(s) may be color independent. For example, ingredients zones 901, 903 on the first side of the article 900A may have the same color while the ingredient zone 905 on the second side of the article 900B may have the same or a different color than ingredient zones 901, 903.

In another embodiment, the color(s) of the article may indicate the type of relief associated with the article. For example, an article containing an ingredient for sore throat relief may have the color red while an article having an ingredient for a cough suppressant may have the color green. The color of the article may also represent a combination of active ingredients. For example, an article having ingredients for an anti-histamine and decongestant may be purple. The colors may also be associated with a flavor of the article. For example, the color red may be associated with a cherry flavor, the color green may be associated with an apple flavor and the color purple may be associated with a grape flavor. In alternate embodiments, any suitable colors, flavors, arrangement and combinations thereof may be utilized.

The articles described herein may be manufactured in any suitable manner including a slot die that has different formulas being coated on the article in different or distinct stripes positioned substantially adjacent or next to each other. In alternate embodiment the article may be coated using knife-over-roll with baffles behind the knife that would keep the different liquids separated until they pass under the knife. In other alternate embodiments a formula or solution may be applied to the article by, for example, spraying, dipping, comma coaters, powder coating, roll over roll, reverse roll.

It should be understood that the foregoing description is only illustrative of the embodiments. Various alternatives and modifications can be devised by those skilled in the art without departing from the embodiments. Accordingly, the present embodiments are intended to embrace all such alternatives, modifications and variances that fall within the scope of the appended claims.

What is claimed is:

1. An article of manufacture comprising:
a substrate having at least one zone on a surface of the substrate; and
at least one ingredient on a surface of the substrate;
wherein the at least one ingredient is associated with a respective one of the at least one zone.

2. The article of claim 1, wherein each of the at least one ingredient is associated with a respective color.

3. The article of claim 1, wherein the at least one ingredient is arranged in stripes on a surface of the substrate.

4. The article of claim 1, wherein the at least one ingredient includes a pharmaceutical agent, flavor and/or odor component.

5. The article of claim 4, wherein at least one odor component releases a fragrance that is complimentary to at least one flavor.

6. The article of claim 1, wherein the article includes a first and second zone where the first and second zones are on one side of the article and divided by a centerline of the article.

7. The article of claim 1, wherein a first side of the article includes at least one zone and a second side of the article includes at least one zone.

8. The article of claim 1, wherein each of the at least one zone includes a different ingredient.

9. The article of claim 1, wherein the article has a color associated with a type of relief provided by the article

10. An article of manufacture comprising:
a substrate; and
at least one ingredient located on a first side of the substrate and at least one ingredient located on a second side of the substrate;
wherein, the fist and second sides have different colors related to an activity level of a respective one of the at least one ingredient.

11. The article of claim 10, wherein each of the first and second sides of the substrate includes at least one zone.

12. The article of claim 11, wherein the at least one ingredient on the first side of the substrate and the at least one ingredient on the second side of the substrate are located in a respective one of the at least one zone.

13. The article of claim 12, where each of the at least one zone includes a different ingredient.

14. The article of claim 10, wherein the at least one ingredient on the first and second sides includes a pharmaceutical agent, flavor and/or odor component.

15. The article of claim 14, wherein at least one odor component releases a fragrance that is complimentary to at least one flavor.

16. An article of manufacture comprising:
a substrate;
at least one active ingredient located on a first side of the substrate; and
at least one ancillary ingredient located on a second side of the substrate;
wherein, when the at least one active ingredient is placed in contact with the at least one ancillary ingredient the at least one active ingredient is activated or an activity of the at least one active ingredient is enhanced.

17. The article of claim 16, wherein the at least one active ingredient and the at least one ancillary ingredient are located in zones.

18. The article of claim 16, wherein the first side of the substrate includes at least two zones where each of the at least two zones contain a different active ingredient.

19. The article of claim 16, wherein the second side of the substrate includes at least two zones where each of the at least two zones contain a different ancillary ingredient.

20. The article of claim 16, wherein each of the at least one active ingredients is associated with a respective color.

21. The article of claim 16, wherein the at least one active ingredient and/or at least one ancillary ingredient includes a pharmaceutical agent flavor and/or odor component.

22. The article of claim 21, wherein at least one odor component releases a fragrance that is complimentary to at least one flavor.
